(19) United States
(12) Patent Application Publication
(10) Pub. No.: US 2021/0369254 A1
(43) Pub. Date: Dec. 2, 2021

(54) MEDICAL FULLY CLOSED PATHOLOGICAL SAMPLING DEVICE

(71) Applicant: China Medical University, Shenyang (CN)

(72) Inventors: RUI ZHAO, Shenyang (CN); DAWEI GUAN, Shenyang (CN); LINLIN WANG, Shenyang (CN)

(21) Appl. No.: 17/160,557

(22) Filed: Jan. 28, 2021

(30) Foreign Application Priority Data
May 28, 2020 (CN) 202010464620.6

(51) Int. Cl. A61B 10/02; A61B 16/00

(52) U.S. Cl. CPC A61B 10/0266; A61B 2010/0208; A61B 16/00

(57) ABSTRACT

A medical fully closed pathological sampling device is disclosed, which addresses the problems existing in the prior art that the size of a sample obtained is too small, which is not conducive to pathological analysis, there is lack of effective closing measures, and environmental pollution and health threats are presented. The device includes an outer sealed cylinder provided with a closable opening at the front end, wherein a middle cutoff cylinder is movably inserted inside the outer sealed cylinder; an inner sampling cylinder is telescopically inserted at the opening located at the front end of the middle cutoff cylinder, and a sampling knife is provided at the front end of the inner sampling cylinder; the front end of a handle inserting rod provided on a sampling operation handle extends into a closable insertion port located at the rear end of the outer sealed cylinder, and the front end of the handle inserting rod is connected to the middle cutoff cylinder and the inner sampling cylinder within the outer sealed cylinder. The device is reasonable in design and compact in structure, and can obtain large-size histological samples, reduce damage to the appearance of cadavers, avoid secondary pollution of the obtained samples to the environment, and effectively reduce the health threat to an anatomy operator and a sample sender.

### FIELD OF THE INVENTION

The present disclosure relates to the technical field of sampling appliances for organ tissues from cadavers of infectious diseases, and particularly to a medical fully closed pathological sampling device that can obtain large-size histological samples, reduce damage to the appearance of the cadavers, avoid secondary pollution of the obtained samples to the environment, and effectively reduce the health threat to an anatomy operator and a sample sender.

### BACKGROUND OF THE INVENTION

Forensic autopsy is to systematically examine the body surface and internal organs of a cadaver, observe whether various organs, tissues and cells have lesions and injuries or not and their extent, and find out the relationship between the lesions of various organs, so as to facilitate determination of the cause of death and clarification of the mechanism of death. Also, the systematic anatomy of cadavers from infectious diseases plays an irreplaceable role in explaining the pathogenic mechanism of infectious disease pathogens and the cause of death of patients. However, the systematic anatomy of infectious disease cadavers is not only restricted by ethics, but also has some problems, such as high requirements for a dissecting room, great threat to the health of autopsy operators, and the possibility of secondary pollution to the environment; and due to the limitations of many factors, the autopsy of infectious disease cadavers is usually not performed in time, which affects the effective prevention and control of infectious diseases and endangers public health. In addition, a biopsy puncture needle currently used in clinical practice, which obtains samples in a small size, is not conducive to a more comprehensive pathological analysis, and the samples are often squeezed; and also, due to the lack of effective closing measures, the obtained samples may cause environmental pollution and personal threat upon being taken out of the body. Therefore, it is necessary to improve the prior art methods and appliances for sampling organ tissues from cadavers of infectious diseases.

### SUMMARY OF THE INVENTION

In view of the above problems, the present disclosure provides a medical fully closed pathological sampling device that can obtain large-size histological samples, reduce damage to the appearance of the cadavers, avoid secondary pollution of the obtained samples to the environment, and effectively reduce the health threat to an anatomy operator and a sample sender.

The technical solution adopted by the present disclosure is that the medical fully closed pathological material sampling device comprises an outer sealed cylinder, wherein a closable opening is provided at the front end of the outer sealed cylinder, and a middle cutoff cylinder is movably inserted inside the outer sealed cylinder; an inner sampling cylinder is telescopically inserted at the opening at the front end of the middle cutoff cylinder, and a sampling knife is provided at the front end of the inner sampling cylinder; a sampling operation handle is provided outside the rear end of the outer sealed cylinder, a handle inserting rod is provided on the sampling operation handle, and the front end of the handle inserting rod extends into a closable insertion port at the rear end of the outer sealed cylinder; and the front end of the handle inserting rod is connected to the middle cutoff cylinder and the inner sampling cylinder inside the outer sealed cylinder.

The outer sealed cylinder is composed of a sealed cylinder main body, a sampling closed cavity is provided inside the sealed cylinder main body, a closed opening is provided at the front end of the sealed cylinder main body, an inserting rod entrance is provided at the rear end of the sealed cylinder main body, the inserting rod entrance is connected to an inserting rod telescoping cavity at the inner rear end of the sampling closed cavity, and a telescoping tube through hole is provided at one end of the inserting rod telescoping cavity that is located inside the sampling closed cavity; the handle inserting rod extends into the inserting rod telescoping cavity via the inserting rod entrance. In this way, the middle cutoff cylinder and the inner sampling cylinder are inserted into the sampling closed cavity of the outer sealed cylinder via the closed opening, and the handle inserting rod is inserted into the inserting rod telescoping cavity through the inserting rod entrance.

The middle cutoff cylinder is composed of a cutoff cylinder main body, a sampling cylinder insertion cavity is provided inside the cutoff cylinder main body, a cylinder insertion port is provided at the front end of the cutoff cylinder main body, a cutoff knife head extending forward is provided on the upper side of the cylinder insertion port; a cutoff cylinder telescoping tube is provided at the rear end of the cutoff cylinder main body, and the rear end of the cutoff cylinder telescoping tube is detachably connected to the front end of the handle inserting rod. In this way, with the use of the sampling cylinder insertion cavity, the middle cutoff cylinder is sleeved outside the inner sampling cylinder, so as to cut off an organ tissue within the inner sampling cylinder by the sheet-like cutoff knife head that can bend and deform.

The inner sampling cylinder is composed of a sampling cylinder main body, a sampling cavity is provided inside the sampling cylinder main body, a sampling inlet is provided at the front end of the sampling cylinder main body, and the sampling knife is provided outside the sampling inlet; a sampling cylinder telescoping rotary rod is provided at the rear end of the sampling cylinder main body, and the sampling cylinder telescoping rotary rod is inserted into a telescoping rotary rod insertion port of the cutoff cylinder telescoping tube located at the rear end of the middle cutoff cylinder; and a cutoff knife insertion opening, which facilitates the insertion of the cutoff knife head at the front end of the middle cutoff cylinder, is provided on the sidewall at the front end of the sampling cylinder main body. In this way, the organ tissue is cut by the sampling knife, and the cut organ tissue is allowed to enter into the cylinder shaped sampling cavity via the sampling inlet; and then the sheet-like cutoff knife head that can bend and deform, which is entered into the sampling cavity from the cutoff knife insertion opening, is used to cut off the organ tissue into an organ sample.

A core rod insertion port is provided inside the handle inserting rod, a rotary rod connecting core rod is provided within the core rod insertion port, and the rotary rod connecting core rod extends to a rotary rod connection at the front end inside the core rod insertion port, and is detachably connect to a core rod connection at the rear end of the sampling cylinder telescoping rotary rod of the inner sampling cylinder; and, a core rod positioning flange is provided at one end of the rotary rod connecting core rod that is located outside the sampling operation handle, and the core rod positioning flange is detachably connected at a fixed position to the sampling operation handle. In this way, the rotary rod connecting core rod connected at a fixed position to the sampling operation handle is used to connect the handle inserting rod to the sampling cylinder telescoping rotary rod of the inner sampling cylinder, facilitating the operation of the inner sampling cylinder.

A gripping projection that facilitates the insertion and removal operations of the connecting core rod is provided on the outer side of the core rod positioning flange of the rotary rod connecting core rod, and a screw hole is also provided on the core rod positioning flange; a core rod fixation screw passes through the screw hole and is connected to a core rod fixation screw hole on the sampling operation handle that is provided alongside the outer end of the core rod insertion port. In this way, the rotary rod connecting core rod inserted inside the handle inserting rod is connected at a fixed position to the sampling operation handle, and thus the rotary rod connecting core rod as well as the sampling cylinder telescoping rotary rod and the inner sampling cylinder connected to the end thereof are driven by the sampling operation handle to telescope or rotate together.

The sampling knife provided outside the sampling inlet at the front end of the inner sampling cylinder is an annular sampling knife, and several sets of circumferentially arranged serrated cutting blades are provided on the annular sampling knife. In this way, the rotation and telescoping of the annular sampling knife at the front end of the inner sampling cylinder is used to cut off the organ tissue along the inner wall of the cylinder.

A cutoff cylinder connection engaging block is provided on the sampling cylinder telescoping rotary rod of the inner sampling cylinder at one end close to the sampling cylinder main body; accordingly, a sampling cylinder connection engaging claw is provided on the bottom surface inside the sampling cylinder insertion cavity of the middle cutoff cylinder, and an engaging block engaging port that facilitates the engagement of the cutoff cylinder connection engaging block is provided at the front end of the sampling cylinder connection engaging claw. In this way, after performing an annular cutting of the organ tissue by the inner sampling cylinder, the handle inserting rod continues to be pushed forward, so that the cutoff knife head at the front end of the middle cutoff cylinder is extended into the sampling cavity of the inner sampling cylinder via the cutoff knife insertion opening, so as to cut off the annularly cut organ tissue within the sampling cavity; at the same time, the mated engagement of the sampling cylinder connection engaging claw within the sampling cylinder insertion cavity of the middle cutoff cylinder with the cutoff cylinder connection engaging block on the sampling cylinder telescoping rotary rod of the inner sampling cylinder, is used to connect the inner sampling cylinder and the middle cutoff cylinder together; as such, it is convenient to subsequently pull out the handle inserting rod by the sampling operation handle in the opposite direction, so as to retract the inner sampling cylinder together with the middle cutoff cylinder back into the sampling closed cavity of the outer sealed cylinder.

An axially arranged cutoff guide slot is provided on the sidewall of the cutoff cylinder main body of the middle cutoff cylinder, and a cutoff guide projection is provided outside the sidewall of the sampling cylinder main body of the inner sampling cylinder at a location corresponding to the cutoff guide slot on the middle cutoff cylinder. In this way, the guide on the forward telescoping of the middle cutoff cylinder sleeved outside the inner sampling cylinder is provided through the mated guide connection of the cutoff guide slot and the cutoff guide projection, so as to ensure that the cutoff knife head at the front end of the middle cutoff cylinder can smoothly extend into the cutoff knife insertion opening on the side wall of the inner sampling cylinder.

A sampling cylinder exhaust hole is provided on the bottom surface inside the sampling cavity of the inner sampling cylinder, and an ultra-filtration membrane used to filter out pathogens is provided inside the bottom surface of the sampling cavity. In this way, while the sampling cylinder exhaust hole is used to exhaust excessive gas within the sampling cavity during sampling, the ultra-filtration membrane is used to filter out pathogens possibly present in the exhausted gas, thereby improving the safety of the device in use.

The beneficial effects of the present disclosure is that since the present utility adopts a structural form: an outer sealed cylinder provided with a closable opening at the front end, wherein a middle cutoff cylinder is movably inserted inside the outer sealed cylinder; an inner sampling cylinder is telescopically inserted at the opening located at the front end of the middle cutoff cylinder, and a sampling knife is provided at the front end of the inner sampling cylinder; a sampling operation handle is provided outside the rear end of the outer sealed cylinder, the front end of a handle inserting rod provided on a sampling operation handle extends into a closable insertion port located at the rear end of the outer sealed cylinder, and the front end of the handle inserting rod is connected to the middle cutoff cylinder and the inner sampling cylinder within the outer sealed cylinder, it is reasonable in design and compact in structure, and can obtain samples of various organs of the dead through extracorporeal puncture, and conduct pathological examination and analysis, to ensure that the size of the extracted tissue mass can meet the requirements of forensic examination, which is conducive to a comprehensive and systematic understanding of pathological damage to organ tissues caused by infectious pathogens. Moreover, the device reduces damage to the appearance of cadavers, and only requires that perforation is performed on the body surface and that after the sample is taken, the perforation site is filled with an absorbent material followed by suturing closure, which can effectively prevent blood outflow from the cadavers; and the device reduces large exposure of open wounds in the traditional autopsy and secondary environmental pollution caused by the blood outflow from the cadavers. In addition, it can protect the health and safety of a dissection operator, and prevent the operator from being exposed to a high-risk environment of possible high concentrations of pathogens in the entire process; moreover, the fully closed sampling method of the device can guarantee a full closed transfer for the whole process from sampling from the cadaver to operating of the sample in the lab, and effectively reduce the health threat to a dissection operator and a sample sender, which has important forensic and medical significance.

### BRIEF DESCRIPTION OF THE DRAWINGS

FIG. 1 is a schematic diagram of a structure of the present disclosure.

FIG. 2 is a cross sectional view of the internal structure of FIG. 1.

FIG. 3 is an exploded schematic diagram of the structure of FIG. 1.

FIG. 4 is a schematic diagram of a structure of an inner sampling cylinder in FIG. 3.

FIG. 5 is a cross sectional view of the internal structure of FIG. 4.

FIG. 6 is a schematic diagram of a structure of a middle cutoff cylinder in FIG. 3.

FIG. 7 is a cross sectional view of the internal structure of FIG. 6.

FIG. 8 is a cross sectional view of the internal structure of an outer sealed cylinder in FIG. 3.

FIG. 9 is a schematic diagram of a connecting structure of a sampling operation handle and a handle inserting rod in FIG. 3.

FIG. 10 is a schematic diagram of a structure of a rotary rod connecting core rod in FIG. 3.

FIG. 11 is a front view of FIG. 2 (enlarged).

FIGS. 12-16 are schematic diagrams of stepwise actions during the use of the present disclosure.

Description of serial numbers in the figures: 1 outer sealed cylinder, 2 inserting rod telescoping cavity, 3 handle inserting rod, 4 sampling operation handle, 5 sampling closed cavity, 6 inner sampling cylinder, 7 annular sampling knife, 8 sampling cylinder telescoping rotary rod, 9 middle cutoff cylinder, 10 cutoff cylinder telescoping tube, 11 rotary rod connecting core rod, 12 core rod fixation screw, 13 core rod insertion port, 14 cutoff guide slot, 15 cutoff knife head, 16 cutoff guide projection, 17 cutoff knife insertion opening, 18 sampling cylinder exhaust hole, 19 sampling cylinder main body, 20 core rod connection, 21 cutoff cylinder connection engaging block, 22 sampling cavity, 23 sampling inlet, 24 absorbent sponge, 25 ultra-filtration membrane, 26 cutoff cylinder main body, 27 telescoping rotary rod insertion port, 28 sampling cylinder insertion cavity, 29 cylinder insertion port, 30 sampling cylinder connection engaging claw, 31 engaging block engaging port, 32 sealed cylinder main body, 33 closed opening, 34 inserting rod entrance, 35 telescoping tube through hole, 36 sealed cylinder exhaust hole, 37 core rod fixation screw hole, 38 rotary rod connection, 39 core rod positioning flange, 40 screw hole, 41 gripping projection, 42 organ tissue, 43 organ sample, 44 inserting rod port plug, 45 closed port plug.

### DETAILED DESCRIPTION OF THE INVENTION

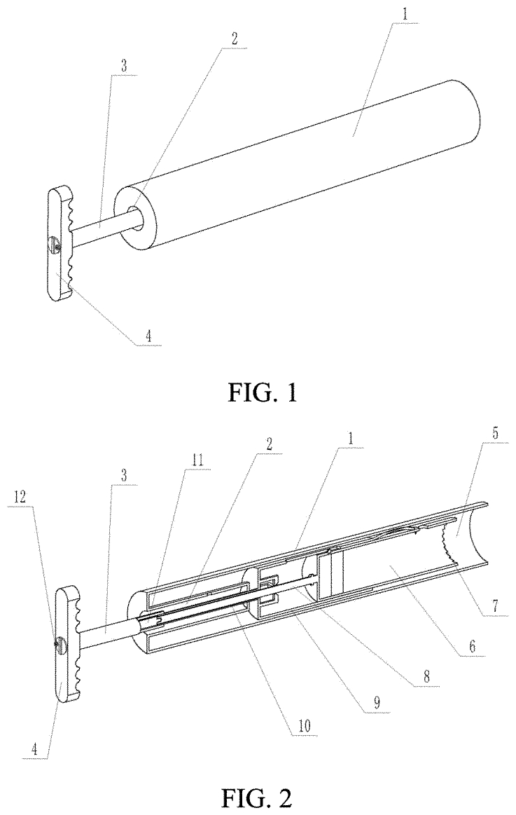

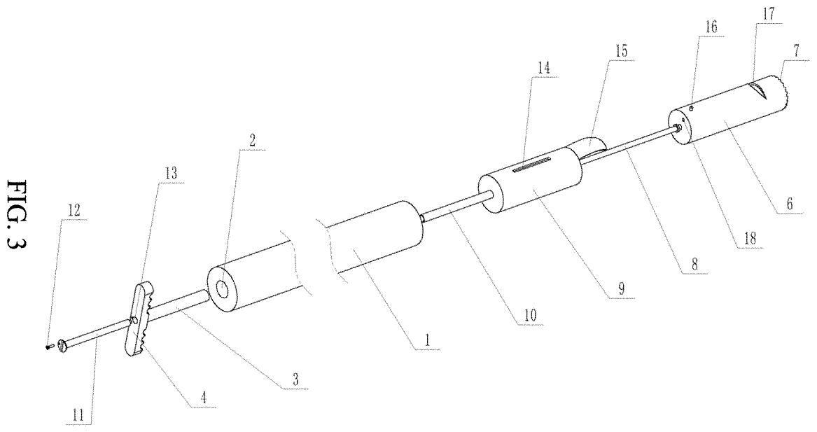

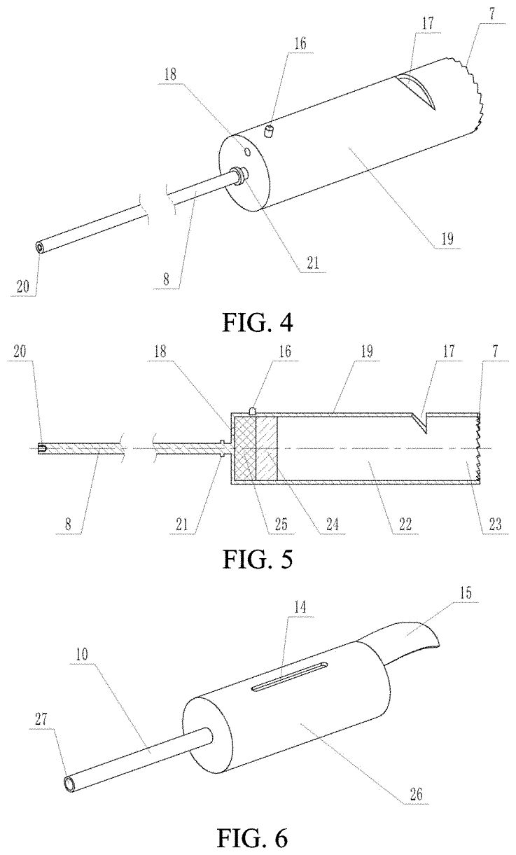

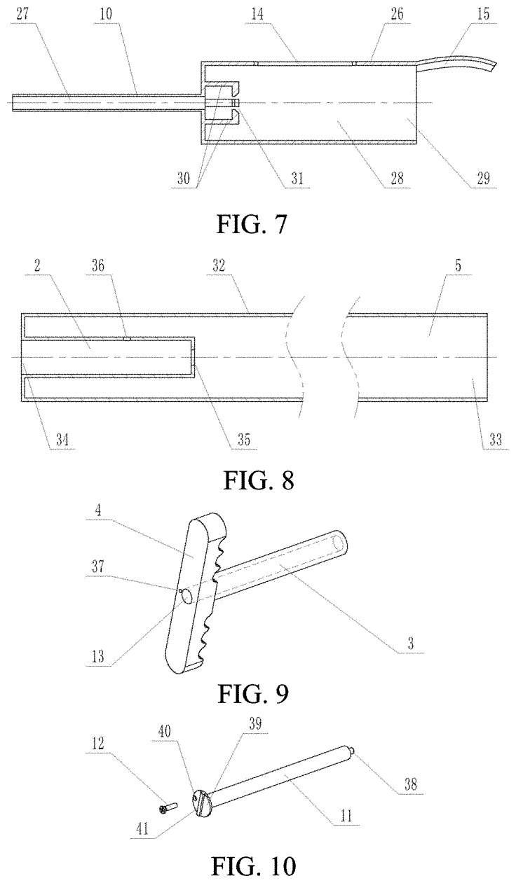

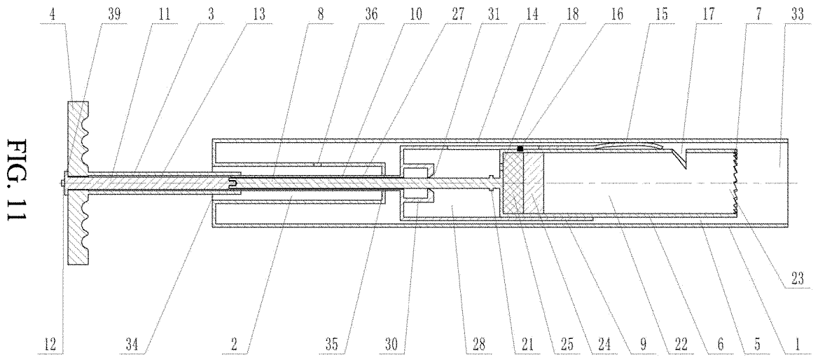

A specific structure of the present disclosure is described in detail with reference to FIGS. 1-11. The medical fully closed pathological sampling device includes an outer sealed cylinder 1, and the outer sealed cylinder 1 is composed of a sealed cylinder main body 32. The interior of the sealed cylinder main body 32 is provided with a sampling closed cavity 5, and the front end of the sealed cylinder main body 32 is provided with a closed opening 33, and a detachable closing plug 45 is provided at the closed opening 33. The rear end of the sealed cylinder 32 is provided with a inserting rod entrance 34, the inserting rod entrance 34 is connected to an inserting rod telescoping cavity 2 disposed at the inner rear of the sampling closed cavity 5, and a sealed cylinder exhaust hole 36 is provided on the side wall of the inserting rod telescoping cavity 2. The inserting rod telescoping cavity 2 is located at one end inside the sampling closed cavity 5, and is provided with a telescoping tube through hole 35 for passage of a cutoff cylinder telescoping tube 10 of a middle cutoff cylinder 9; a handle inserting rod 3 connected to a sampling operation handle 4 extends into the inserting rod telescoping cavity 2 via the inserting rod entrance 34, so as to insert, via the closed opening 33, the middle cutoff cylinder 9 and an inner sampling cylinder 6 into the sampling closed cavity 5 of the outer sealed cylinder 1.

The middle cutoff cylinder 9 is movably inserted into the sampling closed cavity 5 of the outer sealed cylinder 1. The middle cutoff cylinder 9 is composed of a cutoff cylinder main body 26, and a sampling cylinder insertion cavity 28 is provided in the interior of the cutoff cylinder main body 26. A cylinder insertion port 29 is provided at the front end of the cutoff cylinder main body 26. A forward-extending bendable sheet-like cutoff knife head 15 is provided on the upper side of the cylinder insertion port 29. A cutoff cylinder telescoping tube 10 is provided at the rear end of the cutoff cylinder main body 26. A telescoping rotary rod insertion port 27, which is used for the insertion of a sampling cylinder telescoping rotary rod 8 of the inner sampling cylinder 6, is provided in the interior of the cutoff cylinder telescoping tube 10 and through the cutoff cylinder telescoping tube 10. And, the rear end of the cutoff cylinder telescoping tube 10 is threadedly and detachably connected to the front end of the handle inserting rod 3 that extends into the inserting rod telescoping cavity 2, so that the handle inserting rod 3 together with the cutoff cylinder telescoping tube 10 and the middle cutoff cylinder 9 connected to the end thereof are driven by the sampling operation handle 4 to telescope or rotate. As a result, with the use of the sampling cylinder insertion cavity 28, the middle cutoff cylinder 9 is sleeved outside the inner sampling cylinder 6, so as to cut off an organ tissue 42 within the inner sampling cylinder 6 by the sheet-like cutoff knife head 15 that can bend and deform.

The inner sampling cylinder 6 is telescopically inserted at the cylinder insertion port 29 located at the front end of the middle cutoff cylinder 9, the inner sampling cylinder 6 is composed of a sampling cylinder main body 19, and a sampling cavity 22 for housing an organ sample 43 is provided in the interior of the sampling cylinder main body 19. A sampling inlet 23 is provided at the front end of the sampling cylinder main body 19, an annular sampling knife 7 is provided outside the sampling inlet 23, and several sets of circumferentially arranged serrated cutting blades are provided on the annular sampling knife 7, so as to use the rotation and telescoping of the annular sampling knife 7 located on the front end of the inner sampling cylinder 6 to cut off the organ tissue 42 along the inner wall of the cylinder. A sampling cylinder exhaust hole 18 is provided on the bottom surface inside the sampling cavity 22 of the inner sampling cylinder 6, and a ultra-filtration membrane 25 (having a rated aperture range of less than 0.02 microns) used to filter out pathogens is provided inside the bottom surface of the sampling cavity 22. In addition, an absorbent sponge 24 used for absorbing blood in the organ sample 43 is also provided in the sampling cavity 22 of the inner sampling cylinder 6. As such, while the sampling cylinder exhaust hole 18 is used to exhaust excessive gas within the sampling cavity 22 during sampling, the ultra-filtration membrane 25 is used to filter out pathogens possibly present in the exhausted gas, thereby improving the safety of the device in use.

A sampling cylinder telescoping rotary rod 8 used to drive the telescoping and rotation of the inner sampling cylinder 6 is provided at the rear end of the sampling cylinder main body 19 of the inner sampling cylinder 6; and the sampling cylinder telescoping rotary rod 8 of the inner sampling cylinder 6 is movably inserted into the telescoping rotary rod insertion port 27 of the cutoff cylinder telescoping tube 10 located at the rear end of the middle cutoff cylinder 9. Also, a cutoff knife insertion opening 17 that facilitates the insertion of the cutoff knife head 15 located at the front end of the middle cutoff cylinder 9, is provided on the side wall at the front end of the sampling cylinder main body 19. As such, the organ tissue 42 is cut by the annular sampling knife 7, and the cut organ tissue 42 is allowed to enter into the cylinder shaped sampling cavity 22 via the sampling inlet 23;

and then the sheet-like cutoff knife head 15 that can bend and deform, which is entered into the sampling cavity 22 from the cutoff knife insertion opening 17 of the inner sampling cylinder 6, is used to cut off the organ tissue 42 into a piece of organ sample 43.

The sampling operation handle 4 used to operate the inner sampling cylinder 6 and the middle cutoff cylinder 9, is provided outside the rear end of the outer sealed cylinder 1 at which the inserting rod entrance 34 is arranged; the handle inserting rod 3 is provided on the sampling operation handle 4, and the handle inserting rod 3 extends into the inserting rod telescoping cavity 2 via the inserting rod entrance 34 located at the rear end of the outer sealed cylinder 1. A core rod insertion port 13 arranged through the sampling operation handle 4 and the handle inserting rod 3 is provided inside the handle inserting rod 3, and a rotary rod connecting core rod 11 is provided within the core rod inserting port 13; and, the rotary rod connecting core rod 11 extends into a rotary rod connection 38 located at the inner front end of the core rod insertion port 13 of the handle inserting rod 3, and is threadedly and detachably connected to a core rod connection 20 located at the rear end of the sampling cylinder telescoping rotary rod 8 of the inner sampling cylinder 6. Also, a core rod positioning flange 39 is provided at one end of the rotary rod connecting core rod 11 that is located outside the sampling operation handle 4, and a gripping projection 41 that facilitates the insertion and removal operations of the connecting core rod is provided on the outer side of the core rod positioning flange 39; and a screw hole 40 is also provided on the core rod positioning flange 39. A core rod fixation screw 12 passes through the screw hole 40 and is connected to a core rod fixation screw hole 37 provided on the sampling operation handle 4 alongside the outer end of the core rod insertion port 13; and then the rotary rod connecting core rod 11 inserted into the core rod insertion port 13 of the handle inserting rod 3 is detachably connected at a fixed position to the sampling operation handle 4, and the rotary rod connecting core rod 11 connected to the sampling operation handle 4 at a fixed position is used to connect the handle inserting rod 3 to the sampling cylinder telescoping rotary rod 8 of the inner sampling cylinder 6; as such, the rotary rod connecting core rod 11 together with the sampling cylinder telescoping rotary rod 8 and the inner sampling cylinder 6 connected to the end of the rotary rod connecting core rod 11 are driven by the sampling operation handle 4 to telescope or rotate.

A cutoff cylinder connection engaging block 21 is provided on the sampling cylinder telescoping rotary rod 8 of the inner sampling cylinder 6 at one end close to the sampling cylinder main body 19. Accordingly, an elastic sampling cylinder connection engaging claw 30 is provided on the bottom surface inside the sampling cylinder insertion cavity 28 of the middle cutoff cylinder 9, and the front end of the sampling cylinder connection engaging claw 30 is provided with an engaging block engaging port 31 that facilitates the engagement of the cutoff cylinder connection engaging block 21. As a result, after performing an annular cutting of the organ tissue 42 by the inner sampling cylinder 6, the handle inserting rod 3 continues to be pushed forward, so that the cutoff knife head 15 at the front end of the middle cutoff cylinder 9 is extended into the sampling cavity 22 of the inner sampling cylinder 6 via the cutoff knife insertion opening 17, so as to cut off the annularly cut organ tissue 42 within the sampling cavity 22; at the same time, the mated engagement of the sampling cylinder connection engaging claw 30 within the sampling cylinder insertion cavity 28 of the middle cutoff cylinder 9 with the cutoff cylinder connection engaging block 21 on the sampling cylinder telescoping rotary rod 8 of the inner sampling cylinder 6, is used to connect the inner sampling cylinder 6 and the middle cutoff cylinder 9 together; in this way, it is convenient to subsequently pull out the handle inserting rod 3 by the sampling operation handle 4 in the opposite direction, so as to retract the inner sampling cylinder 6 together with the middle cutoff cylinder 9 back into the sampling closed cavity 5 of the outer sealed cylinder 1.

An axially arranged cutoff guide slot 14 is provided on the sidewall of the cutoff cylinder main body 26 of the middle cutoff cylinder 9, and a cutoff guide projection 16 is provided outside the sidewall of the sampling cylinder main body 19 of the inner sampling cylinder 6 at a location corresponding to the cutoff guide slot 14 on the middle cutoff cylinder 9; and then an effective guide on the forward telescoping of the middle cutoff cylinder 9 sleeved outside the inner sampling cylinder 6 is provided through the mated guide connection of the cutoff guide slot 14 and the cutoff guide projection 16, so as to ensure that the cutoff knife head 15 at the front end of the middle cutoff cylinder 9 can smoothly extend into the cutoff knife insertion opening 17 on the side wall of the inner sampling cylinder 6, to cut off the organ tissue 42 within the sampling cavity 22 of the inner sampling cylinder 6.

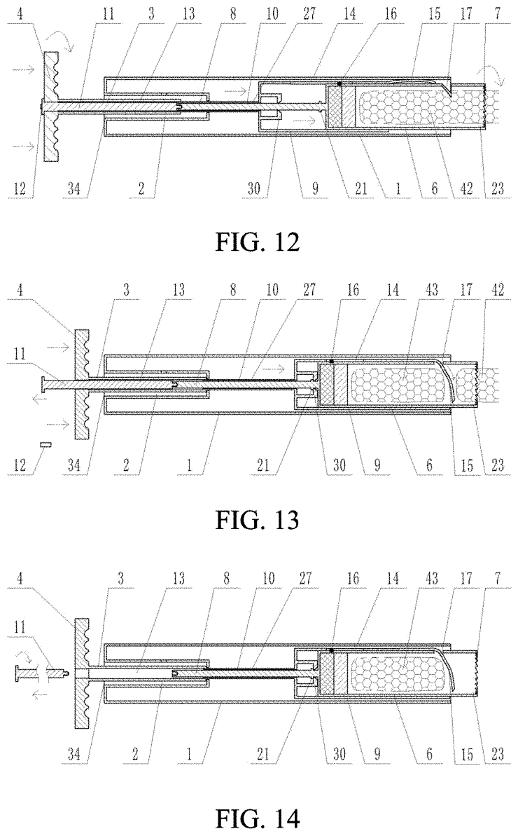

When this medical fully closed pathological sampling device is about to use, in the initial state, the middle cutoff cylinder 9 and the inner sampling cylinder 6 sleeved to each other, are located within the sampling closed cavity 5 of the outer sealed cylinder 1, and the cutoff knife head 15 at the front end of the middle cutoff cylinder 9 is located behind the cutoff knife insertion opening 17 of the inner sampling cylinder 6 (has not yet extended into the cutoff knife insertion opening 17). When a sample of the organ tissue 42 of the infectious disease cadavers needs to be obtained for pathological analysis, firstly, the closed opening 33 at the front end of the outer sealed cylinder 1 is aimed at the skin surface of the cadavers to be sampled; and then, the cutoff cylinder telescoping tube 10 connected to the handle inserting rod 3 as well as the sampling cylinder telescoping rotary rod 8 connected to the rotary rod connecting core rod 11 are driven by the sampling operation handle 4 to carry the middle cutoff cylinder 9 and the inner sampling cylinder 6 respectively to extend forward together. At the same time, the sampling operation handle 4 is turned to carry the middle cutoff cylinder 9 and the inner sampling cylinder 6 inserted at the front end thereof to rotate together, so that the rotation of the annular sampling knife 7 located on the front end of the inner sampling cylinder 6 is used to annularly cut the organ tissue 42, and the annularly cut organ tissue 42 are allowed to gradually enter into the sampling cavity 22 of the inner sampling cylinder 6 (as shown in FIG. 12).

When the length of the organ tissue 42 that is annularly cut off meets the sampling requirement, the extension and rotation of the middle cutoff cylinder 9 and the inner sampling cylinder 6 are stopped, and the core rod fixation screw 12 between the core rod positioning flange 39 of the rotary rod connecting core rod 11 and the sampling operation handle 4 is taken off. After that, the handle inserting rod 3 continues to be pushed forward by using the sampling operation handle 4, to further move the middle cutoff cylinder 9 forward; at this time, since the rotary rod connecting core rod 11 connected to the sampling cylinder telescoping rotary rod 8 has been disconnected from the sampling operation handle 4, and resistance from the cadaver organ tissue 42 exists at the front end of the unrotated inner sampling cylinder 6, the inner sampling cylinder 6 is temporarily stationary relative to the middle cutoff cylinder 9. As such, the cutoff knife head 15 located at the front end of the middle cutoff cylinder 9 that is moving forward then extends into the sampling cavity 22 of the inner sampling cylinder 6 via the cutoff knife insertion opening 17, and thus cut off the annularly cut organ tissue 42 within the sampling cavity 22 into the organ sample 43 (as shown in FIG. 13). At the same time, the mated engagement of the sampling cylinder connection engaging claw 30 of the middle cutoff cylinder 9 and the cutoff cylinder connection engaging block 21 of the inner sampling cylinder 6, is used to connect the inner sampling cylinder 6 and the middle cutoff cylinder 9 as a whole.

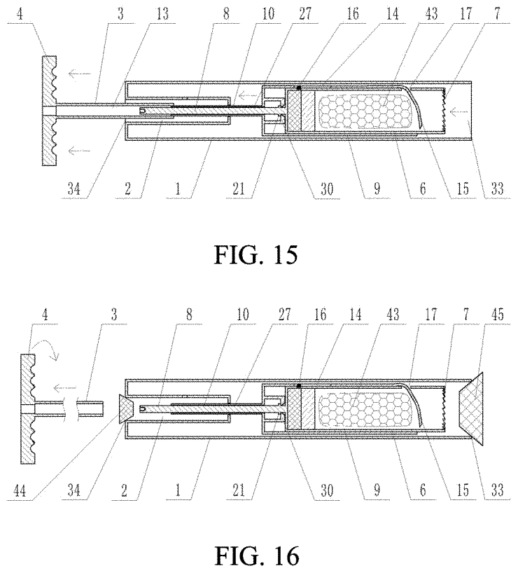

Then, the rotary rod connecting core rod 11 is turned and screwed off, to disconnect the threaded connection with the sampling cylinder telescoping rotary rod 8, and the rotary rod connecting core rod 11 is taken out from the core rod insertion port 13 of the handle inserting rod 3 (as shown in FIG. 14). After that, the handle inserting rod 3 is pulled out by the sampling operation handle 4 in the opposite direction, so that the cutoff cylinder telescoping tube 10 connected to the handle inserting rod 3 is used to pull the middle cutoff cylinder 9 and the inner sampling cylinder 6 that has been connected as a whole to together retract back into the sampling closed cavity 5 of the outer sealed cylinder 1 (as shown in FIG. 15). Subsequently, the sampling operation handle 4 and the handle inserting rod 3 are turned and screwed off, to disconnect the threaded connection with the cutoff cylinder telescoping tube 10, and the handle inserting rod 3 is taken out from the inserting rod telescoping cavity 2; and then, an inserting rod port plug 44 and a closed port plug 45 are used to block the inserting rod entrance 34 and the closed opening 33 at both ends of the outer sealed cylinder 1, respectively (as shown in FIG. 16), so as to ensure a full closed transfer for the whole process from sampling from the cadaver to operating of the sample in the lab.

1. A medical fully closed pathological material sampling device comprising an outer sealed cylinder (1), wherein a closable opening (33) is provided at the front end of the outer sealed cylinder (1), and a middle cutoff cylinder (9) is movably inserted inside the outer sealed cylinder (1); an inner sampling cylinder (6) is telescopically inserted at the opening at the front end of the middle cutoff cylinder (9), and a sampling knife is provided at the front end of the inner sampling cylinder (6); a sampling operation handle (4) is provided outside the rear end of the outer sealed cylinder (1), a handle inserting rod (3) is provided on the sampling operation handle (4), and the front end of the handle inserting rod (3) extends into a closable insertion port at the rear end of the outer sealed cylinder (1); and the front end of the handle inserting rod (3) is connected to the middle cutoff cylinder (9) and the inner sampling cylinder (6) inside the outer sealed cylinder (1).

2. The medical full closed pathological sampling device according to claim 1, wherein the outer sealed cylinder (1) is composed of a sealed cylinder main body (32), a sampling closed cavity (5) is provided inside the sealed cylinder main body (32), a closed opening (33) is provided at the front end of the sealed cylinder main body (32), an inserting rod entrance (34) is provided at the rear end of the sealed cylinder main body (32), the inserting rod entrance (34) is connected to an inserting rod telescoping cavity (2) at the inner rear end of the sampling closed cavity (5), and a telescoping tube through hole (35) is provided at one end of the inserting rod telescoping cavity (2) that is located inside the sampling closed cavity (5); the handle inserting rod (3) extends into the inserting rod telescoping cavity (2) via the inserting rod entrance (34).

3. The medical full closed pathological sampling device according to claim 1, wherein the middle cutoff cylinder (9) is composed of a cutoff cylinder main body (26), a sampling cylinder insertion cavity (28) is provided inside the cutoff cylinder main body (26), a cylinder insertion port (29) is provided at the front end of the cutoff cylinder main body (26), a cutoff knife head (15) extending forward is provided on the upper side of the cylinder insertion port (29); a cutoff cylinder telescoping tube (10) is provided at the rear end of the cutoff cylinder main body (26), and the rear end of the cutoff cylinder telescoping tube (10) is detachably connected to the front end of the handle inserting rod (3).

4. The medical full closed pathological sampling device according to claim 3, wherein the inner sampling cylinder (6) is composed of a sampling cylinder main body (19), a sampling cavity (22) is provided inside the sampling cylinder main body (19), a sampling inlet (23) is provided at the front end of the sampling cylinder main body (19), and the sampling knife is provided outside the sampling inlet (23); a sampling cylinder telescoping rotary rod (8) is provided at the rear end of the sampling cylinder main body (19), and the sampling cylinder telescoping rotary rod (8) is inserted into a telescoping rotary rod insertion port (27) of the cutoff cylinder telescoping tube (10) located at the rear end of the middle cutoff cylinder (9); and a cutoff knife insertion opening (17), which facilitates the insertion of the cutoff knife head (15) at the front end of the middle cutoff cylinder (9), is provided on the sidewall at the front end of the sampling cylinder main body (19).

5. The medical full closed pathological sampling device according to claim 4, wherein a core rod insertion port (13) is provided inside the handle inserting rod (3), a rotary rod connecting core rod (11) is provided within the core rod insertion port (13), and the rotary rod connecting core rod (11) extends to a rotary rod connection (38) at the front end inside the core rod insertion port (13), and is detachably connect to a core rod connection (20) at the rear end of the sampling cylinder telescoping rotary rod (8) of the inner sampling cylinder (6); and, a core rod positioning flange (39) is provided at one end of the rotary rod connecting core rod (11) that is located outside the sampling operation handle (4), and the core rod positioning flange (39) is detachably connected at a fixed position to the sampling operation handle (4).

6. The medical full closed pathological sampling device according to claim 5, wherein a gripping projection (41) that facilitates the insertion and removal operations of the connecting core rod is provided on the outer side of the core rod positioning flange (39) of the rotary rod connecting core rod (11), and a screw hole (40) is also provided on the core rod positioning flange (39); a core rod fixation screw (12) passes through the screw hole (40) and is connected to a core rod fixation screw hole (37) on the sampling operation handle (4) that is provided alongside the outer end of the core rod insertion port (13).

7. The medical full closed pathological sampling device according to claim 4, wherein the sampling knife provided outside the sampling inlet (23) at the front end of the inner sampling cylinder (6) is an annular sampling knife (7), and several sets of circumferentially arranged serrated cutting blades are provided on the annular sampling knife (7).

8. The medical full closed pathological sampling device according to claim 4, wherein a cutoff cylinder connection engaging block (21) is provided on the sampling cylinder telescoping rotary rod (8) of the inner sampling cylinder (6) at one end close to the sampling cylinder main body (19); accordingly, a sampling cylinder connection engaging claw (30) is provided on the bottom surface inside the sampling cylinder insertion cavity (28) of the middle cutoff cylinder (9), and an engaging block engaging port (31) that facilitates the engagement of the cutoff cylinder connection engaging block (21) is provided at the front end of the sampling cylinder connection engaging claw (30).

9. The medical full closed pathological sampling device according to claim 4, wherein an axially arranged cutoff guide slot (14) is provided on the sidewall of the cutoff cylinder main body (26) of the middle cutoff cylinder (9), and a cutoff guide projection (16) is provided outside the sidewall of the sampling cylinder main body (19) of the inner sampling cylinder (6) at a location corresponding to the cutoff guide slot (14) on the middle cutoff cylinder (9).

10. The medical full closed pathological sampling device according to claim 4, wherein a sampling cylinder exhaust hole (18) is provided on the bottom surface inside the sampling cavity (22) of the inner sampling cylinder (6), and an ultra-filtration membrane (25) used to filter out pathogens is provided inside the bottom surface of the sampling cavity (22).
